(19) United States
(12) Patent Application Publication
(10) Pub. No.: US 2012/0016297 A1
(43) Pub. Date: Jan. 19, 2012

(54) Interventional Devices Including Dilute Nanotube-Polymer Compositions, and Methods of Making and Using Same

(76) Inventors: Peter John D'Aquanni, Murrieta, CA (US); Mark C. Bates, Encinitas, CA (US); Kent Stalker, San Marcos, CA (US); Jason Phillips, Wildomar, CA (US)

(21) Appl. No.: 13/051,351

(22) Filed: Mar. 18, 2011

Related U.S. Application Data
(63) Continuation of application No. PCT/US2009/005224, filed on Sep. 18, 2009.
(60) Provisional application No. 61/098,624, filed on Sep. 19, 2008.

(51) Int. Cl. A61M 25/10; B29C 47/08; B29C 65/00; B82Y 30/00; B82Y 40/00

(52) U.S. Cl. 604/96.01; 264/119; 156/196; 977/931; 977/890

(57) ABSTRACT

Under one aspect, an interventional device includes a balloon having a flexible wall. The flexible wall includes a composition including between 0.005 wt. % and 0.20 wt. % of carbon nanotubes dispersed in a polymer. Under another aspect, a method of making an interventional device includes contacting a plurality of polymer particles with a plurality of nanotubes; extruding the polymer particles and the nanotubes to form a composition comprising the polymer and the nanotubes; and blow-casting the composition into a balloon.

### CROSS-REFERENCE TO RELATED APPLICATIONS

This application is a continuation under 35 U.S.C. §120 of International Patent Application No. PCT/US2009/005224, filed Sep. 18, 2009, which claims the benefit of U.S. Provisional Patent Application No. 61/098,624, filed Sep. 19, 2008, the entire contents of each of which are incorporated by reference herein.

### FIELD

This application generally relates to interventional devices such as dilatation catheters.

### BACKGROUND

Percutaneous transluminal coronary angioplasty (PTCA) is a less invasive surgical alternative for patients with vessel narrowing due to atherosclerosis and other diseases and conditions. In a conventional PTCA procedure, a dilatation catheter is inserted into the cardiovascular system, under local anesthesia, to a desired position within the diseased vessel. The catheter generally includes an inflatable balloon formed of a non-porous membrane at its distal end, and means for inflating the balloon. As is illustrated in FIG. 1A, balloon 10 is positioned so that it traverses or crosses stenotic lesion 12 within artery 14. As is illustrated in FIG. 1B, balloon 10 then is inflated with a liquid that compresses balloon 10 against lesion 12, and expands artery 14 in a direction generally perpendicular to its wall, thereby dilating the lumen of the artery.

The mechanical characteristics and size of balloon 10, relative to the mechanical characteristics and size of artery 14 and stenotic lesion 12, affect the success of the PTCA procedure. As is known in the art, balloons are typically fabricated so as to be either “non-compliant,” “semi-compliant,” or “compliant.” Non-compliant balloons are constructed of a material that can withstand higher pressures than semi-compliant and compliant balloons, and that allows the diameter of the balloon to expand by between 0-10% above nominal when pressurized. Semi-compliant balloons are constructed of a material that can withstand higher pressures than compliant balloons, and that allows the diameter of the balloon to expand by between 10-20% above nominal when pressurized. Compliant balloons are constructed of a material that allows the diameter of the balloon to expand by greater than 20% above nominal when pressurized (e.g., between 20-200%) but cannot withstand pressures as high as non-compliant or semi-compliant materials.

The physician typically selects the size of balloon 10 based on the estimated size of artery 14, and selects the type of balloon 10 based on the estimated mechanical characteristics of stenotic lesion 12. For example, the physician may select a 3 mm diameter balloon formed of a semi-compliant material to attempt to open an artery occluded by a lesion of average mechanical strength. However, during the procedure, the physician may discover that the size and type of the balloon is actually inappropriate for the artery and lesion, resulting in only partial restoration of the patency of the artery. For example, as illustrated in FIG. 1B, balloon 10 is formed of a non-compliant or semi-compliant material. Although such a material may be useful for disrupting harder lesions 12, it constrains the amount by which the diameter of the balloon can be increased using pressurization. In FIG. 1B, balloon 10 has sufficient mechanical strength that a sufficient pressure may be applied to disrupt lesion 12, but is insufficiently sized to completely restore patency to artery 14. Therefore, the physician may need to exchange the catheter for another having a differently sized balloon 10. Or, for example, as illustrated in FIG. 1C, balloon 10 may be formed of a compliant material. Even if the artery is larger than the physician anticipates, the use of such a material may allow balloon 10 to be sufficiently expanded to restore patency to artery 14. However, if an unexpectedly high pressure is needed to disrupt lesion 12, the application of such a pressure to balloon 10 may overinflate the balloon and over-expand artery 14, which may cause damage (illustrated by the wavy lines in FIG. 1C). Alternately, compliant balloon 10 may rupture when overinflated, causing dissection of the vessel.

In view of the foregoing, it would be desirable to provide an interventional device having flexible walls with improved mechanical properties.

### SUMMARY

The present invention provides an interventional device, such as a balloon catheter suitable for PTCA, having flexible walls with improved mechanical properties.

In accordance with one aspect of this invention, an interventional device includes a balloon having a flexible wall. The flexible wall includes a composition including between 0.005 wt. % and 0.20 wt. % of carbon nanotubes dispersed in a polymer.

In some embodiments, the interventional device comprises an elongated shaft having proximal and distal ends and a lumen therebetween, wherein the balloon is affixed to the elongated shaft near the distal end. In some embodiments, at least a subset of the nanotubes contact other nanotubes to foam a reinforcing web through the flexible wall of the balloon. In some embodiments, the nanotubes are randomly oriented relative to an orientation of the balloon. The nanotubes may be substantially evenly dispersed throughout the composition. The polymer may comprise, for example, nylon, PEBAX®, polyurethane, silicone, PET, or polyethylene. In some embodiments, the balloon has a wall thickness of less than 0.0005″ and a burst pressure of greater than 16 atm. In some embodiments, the wall has a thickness of less than 1/250 of a nominal diameter of the balloon.

In alternative embodiments, the composition from which the balloon is formed comprises between 0.05 wt. % and 0.2 wt. % of the nanotubes, or between 0.1 wt. % and 0.2 wt. % of the nanotubes. In some embodiments, the balloon has an electrical conductivity of at least 3×10⁻¹⁶ S/cm, or at least 6×10⁻⁹ S/cm.

In accordance with another aspect of the invention, a method of making an interventional device includes contacting a plurality of polymer particles with a plurality of nanotubes; extruding the polymer particles and the nanotubes to form a composition comprising the polymer and the nanotubes; and forming the composition into a balloon.

In some embodiments, the nanotubes are in the form of a powder, and the polymer particles and the nanotubes are agitated together to adhere the nanotubes to surfaces of the particles. Alternatively, the nanotubes may be suspended in a liquid that is contacted with the polymer particles. In other embodiments, the nanotubes are aerosolized, the polymer particles are sprayed into the aerosol. In some embodiments, the composition comprises between 0.005 wt. % and 0.2 wt. % of nanotubes. The size of the polymer particles may be selected to provide a desired degree of interconnectedness of the nanotubes in the balloon. For example, the size of the polymer particles may be selected such that the balloon has an electrical conductivity of at least 3×10⁻¹⁶ S/cm, or at least 6×10⁻⁹ S/cm. The polymer used in the composition may comprise any suitable biocompatible polymer such as are conventionally used for balloon catheters, such as nylon, PEBAX®, polyurethane, polyethylene, silicone, or PET. In some embodiments, the material may be crosslinked using electromagnetic (e.g., e-beam) irradiation to further improve its properties.

### BRIEF DESCRIPTION OF THE DRAWINGS

The above and other aspects of the invention will be apparent upon consideration of the following detailed description, taken in conjunction with the accompanying drawings, in which like reference characters refer to like parts throughout, and in which:

FIG. 1A illustrates a cross-sectional view of a deflated, conventional balloon positioned adjacent a stenotic lesion in an artery.

FIG. 1B illustrates a cross-sectional view of an inflated, non-compliant or semi-compliant conventional balloon insufficiently compressing a stenotic lesion in an artery.

FIG. 1C illustrates a cross-sectional view of an inflated, compliant conventional balloon over-expanding an artery.

FIG. 2A illustrates a high-level plan view of an illustrative dilatation catheter of the present invention that includes a balloon formed of a dilute nanotube-polymer composition.

FIG. 2B illustrates a longitudinal sectional view of the balloon of FIG. 2A.

FIG. 2C illustrates a longitudinal sectional view of an alternative embodiment of the balloon of FIG. 2A.

FIG. 2D illustrates a plan view of an alternative embodiment of the balloon of FIG. 2A.

FIG. 3 is a flow chart describing steps of an illustrative method of restoring patency to an occluded artery using a balloon including a dilute nanotube-polymer composition.

FIG. 4A is a flow chart describing illustrative steps of a method of forming the balloon of FIGS. 2A-2C.

FIG. 4B is a flow chart describing illustrative steps of an alternative method of forming the balloon of FIGS. 2A-2C.

FIG. 5 is a cross-sectional illustration of a balloon folded around a catheter shaft.

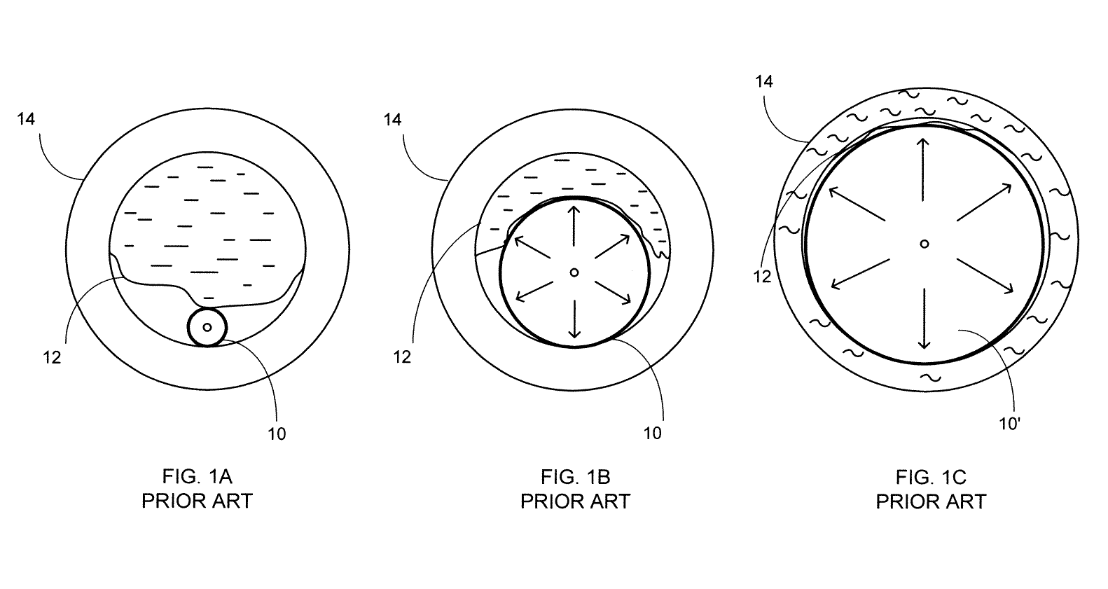

### DETAILED DESCRIPTION OF THE PREFERRED EMBODIMENTS

The present invention is directed to interventional devices including balloons formed from dilute nanotube-polymer compositions, and methods of making and using same. The devices of the present invention may be used for PTCA, stent delivery, or any of a variety of applications employing balloon catheters.

Specifically, embodiments of the invention provide balloons having a flexible wall made of a dilute nanotube-polymer composition. In this specification, the phrase “dilute nanotube-polymer composition,” means a composition that includes a polymer matrix with a plurality of carbon nanotubes dispersed therein, in which the nanotubes are present in a concentration of less than about 0.5% by weight of the composition. The carbon nanotubes form a strong, reinforcing web within the composition, thus enhancing the mechanical properties of the composition from which the balloon wall is fabricated. Among other things, nanotubes have a high tensile strength and elastic modulus, which increases the mechanical strength of the composition. This allows a balloon wall to be fabricated having reduced dimensions and/or capable of withstanding higher pressures than would be possible in an otherwise identical balloon that lacks nanotubes. The concentration of nanotubes, the polymer in which the nanotubes are dispersed, and the thickness to which the balloon wall is fabricated preferably are co-selected to yield a balloon having desired size and mechanical characteristics. The balloon thus fabricated may be used to restore patency to arteries of many sizes that are occluded by lesions having many different mechanical properties. For example, in one embodiment, the loading of nanotubes in a 3 mm nominal diameter balloon may be 0.1% for a given wall thickness of 0.0005″, with a desired rated burst pressure of at least 16 atm. In another embodiment, the loading of nanotubes in a 3 mm nominal diameter balloon may be less than 0.1% for a given wall thickness less than 0.0005″, with a desired rated burst pressure slightly less than 16 atm. In some embodiments, the wall has a thickness of less than 1/250 of a nominal diameter of the balloon.

Among other things, the improved mechanical properties of balloons formed using a dilute nanotube-polymer composition allow the walls of such balloons to be fabricated with a reduced thickness as compared to an otherwise identical balloon lacking nanotubes. By reducing the thickness of the balloon wall, the cross-sectional profile of the deflated balloon can be reduced, thus enhancing the physician's ability to manipulate and position the balloon prior to inflation. For example, the balloon may achieve a reduced delivery profile, thus permitting the catheter to be introduced into arteries that are too small to be accessed using conventional balloons, e.g., intracranial arteries. Or, for example, the pressure to which a balloon formed using a dilute nanotube-polymer composition may be inflated is significantly higher than that of an otherwise identical balloon lacking nanotubes. Conventionally, achieving higher rated burst pressure for balloons without nanotubes may be done by increasing the balloon wall thickness. However, the increased wall thickness may lower the compliance of the conventional balloon, increase the profile (size) of the collapsed and folded balloon, and negatively impact the balloon and adjoining catheter's ability to track through tortuous anatomy. In contrast, a balloon formed using the dilute nanotube-polymer may be employed to disrupt stenotic lesions that cannot be treated using an otherwise similar balloon formed only of a polymer. Moreover, balloons formed using dilute-nanotube compositions are expected to burst within a far narrower range of pressures than otherwise identical, conventional balloons lacking nanotubes. That is, the standard deviation of burst pressures of the balloons made in accordance with the present invention is expected to be significantly lower than that of conventional balloons, making the operation of the balloons, particularly at higher pressures, more reliable than conventional balloons.

Without wishing to be bound by a theory, it is believed that nanotubes in a nanotube-polymer composition experience two competing attractive forces: a van der Waals force that attracts nanotubes to each other, and an attraction between the nanotubes and the polymer, with the van der Waals force being the stronger of the two. At low concentrations, the nanotubes may be spaced, on average, a sufficient distance apart that the attractive van der Waals force is insufficiently strong to cause significant agglomeration of the nanotubes. This is believed to allow the nanotubes to substantially evenly disperse throughout the polymer, forming a “web” that strengthens the balloon wall. Interconnections between the nanotubes may be established, or enhanced, when the balloon is formed using the composition. Specifically, when the composition is stretched to form the thin wall of the balloon (e.g., using blow casting) nanotubes that did not previously contact each other, or for that matter may not have even been near each other, may be brought into contact with each other, thus forming an interconnected network that enhances the mechanical strength of the wall of the balloon.

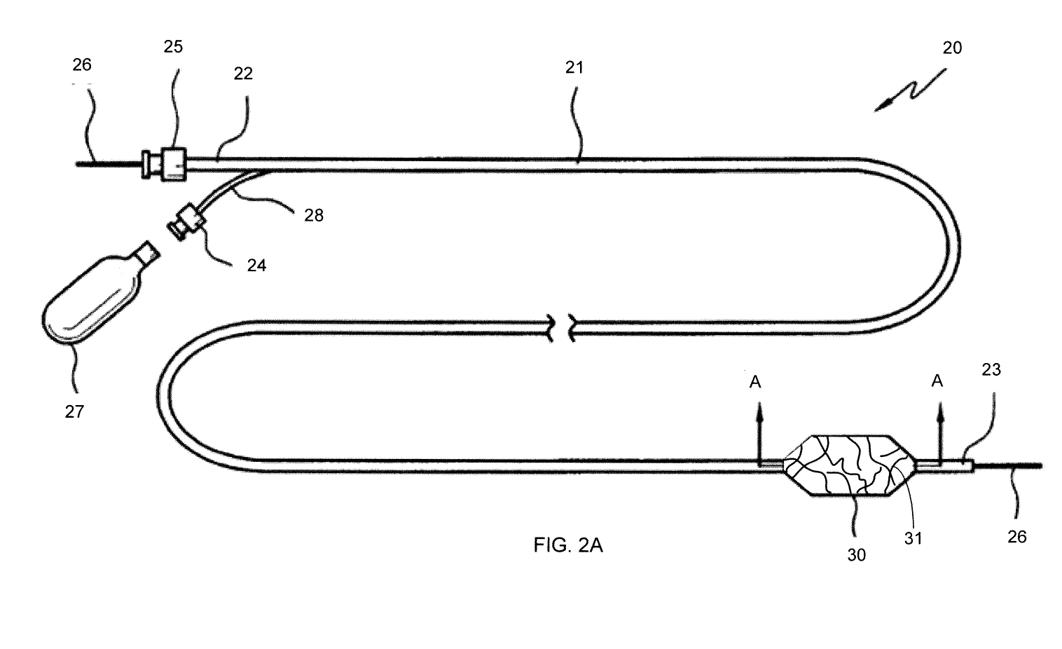

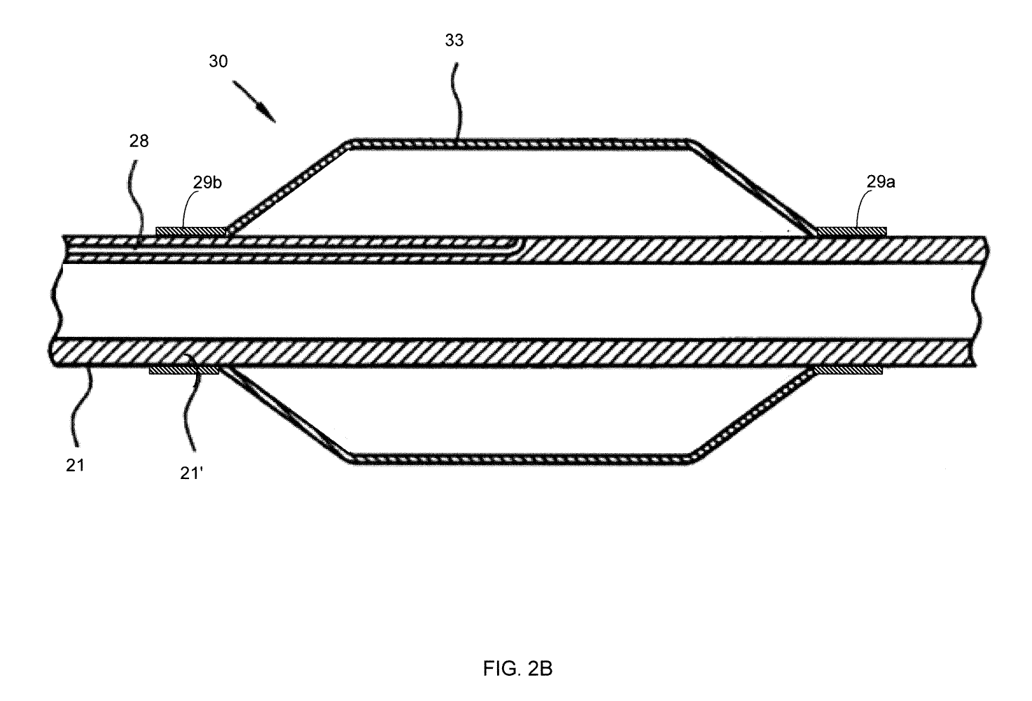

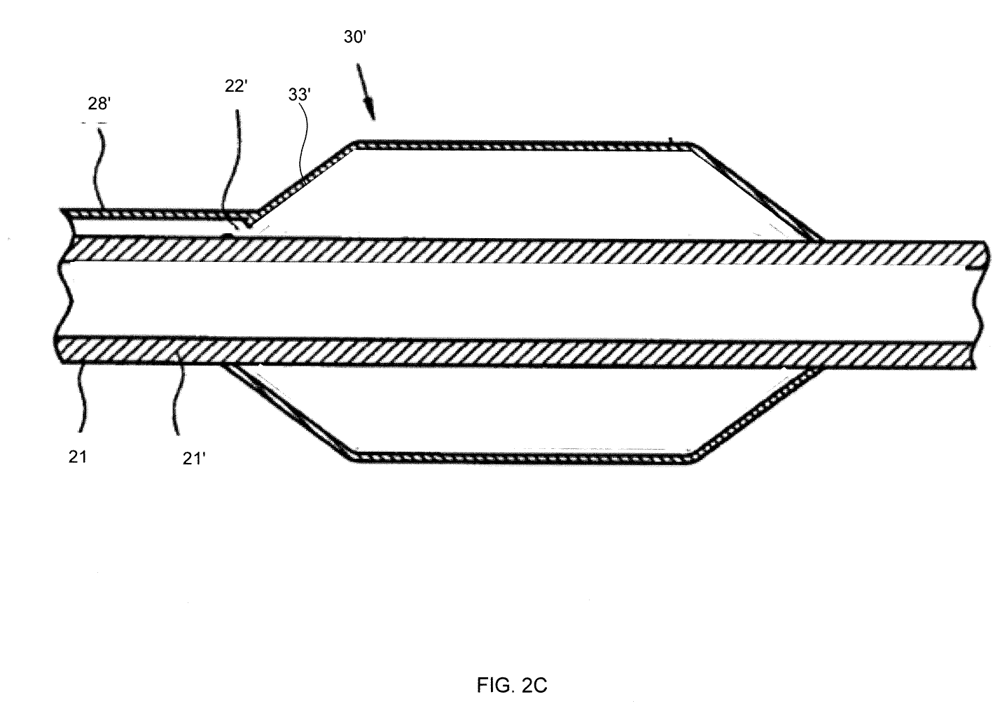

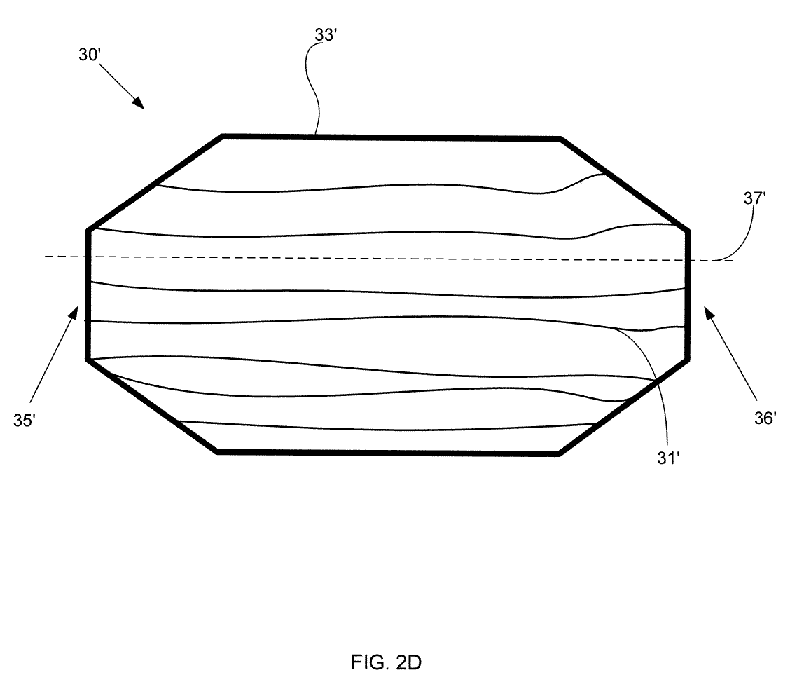

FIG. 2A illustrates a high-level plan view of a dilatation catheter constructed in accordance with some embodiments of the present invention. Catheter 20 includes shaft 21, proximal end 22, distal end 23, inflation port 24, manifold 25, guide wire 26, inflator 27, inflation lumen 28, and balloon 30 formed of a dilute nanotube-polymer composition. Inflator 27 is in fluidic communication with balloon 30 through inflation lumen 28 and may include a syringe or pump as is conventional for use with interventional devices. Inflator 27 supplies a pressurized liquid, e.g., a contrast agent that inflates balloon 30 and allows the dilatation catheter to be imaged in situ using radiography.

As illustrated in greater detail in FIG. 2B, nanotube-reinforced balloon 30 includes flexible wall 33 affixed to shaft 21 via distal and proximal affixation zones 29a and 29b respectively. Inflation lumen 28 passes through wall 21′ of shaft 21, so that a distal end of inflation lumen 28 communicates with the space defined between the outer surface of shaft 21 and the inner surface of the flexible wall 33. The proximal end of inflation lumen 28 is coupled to inflation port 24. Inflation port 24 is coupled to inflator 27 (not shown in FIG. 2B).

Flexible wall 33 of balloon 30 includes a composition that includes a plurality of carbon nanotubes 31 that are dispersed in a polymer. Nanotubes 31 are “dilute” in the polymer, meaning that the nanotubes 31 present at low concentrations in the composition, e.g., at a concentration of less than 0.5 wt. % in the composition. In accordance with various embodiments of the present invention, nanotubes 31 may be present in a concentration of or less than 0.2 wt. %, or between 0.5 wt. % to 0.001 wt. %, or between 0.5 wt. % to 0.01 wt. %, or between 0.25 wt. % to 0.001 wt. %, or between 0.20 wt % to 0.001 wt. %, or between about 0.20 wt. % to 0.01 wt. %, or between 0.20 wt. % to 0.05 wt. %, or between 0.20 wt. % to 0.1 wt. %, or between 0.15 wt. % to 0.05 wt. %, or between 0.2 wt % to 0.15 wt. %, or between 0.15 wt % to 0.10 wt. %, or between 0.10 wt. % to 0.05 wt. %, or about 0.2 wt %, or about 0.15 wt. %, or about 0.1 wt. %, or about 0.05 wt %, or about 0.025 wt %, or about 0.01 wt %, in the composition. For example, nanotubes 31 may be present in a concentration of between 0.2 wt % and 0.01 wt. % in the composition. Or, for example, nanotubes 31 may be present in a concentration of between 0.2 wt. % and 0.05 wt. % in the composition. Or, for example, nanotubes 31 may be present in a concentration of between 0.15 wt. % and 0.05 wt % in the composition. Other concentrations, and ranges of concentrations, are also contemplated. As illustrated further below in the section entitled “Examples,” balloons that are formed from compositions including dilute concentrations of nanotubes in a polymer have surprisingly improved characteristics relative to balloons that are formed from compositions that include high concentrations of nanotubes in a polymer, e.g., concentrations greater than 1 wt. % of nanotubes.

In some embodiments, the composition from which wall 33 is formed consists essentially of nanotubes 31 and polymer, so the weight percent of nanotubes relative to the composition is the same as the weight percent of nanotubes relative to the polymer. Those of skill in the art will recognize that small amounts of impurities may be present in the nanotubes and/or polymer prior to mixing, such as residual catalyst, amorphous carbon, polymer initiators, and the like. Even if impurities are present in the nanotubes and/or polymer, a composition in which only such nanotubes and such polymer are mixed together is still considered to consist essentially of nanotubes and polymer. In other embodiments, other compounds are present at low levels in the composition. For example, as described in greater detail below, in some embodiments a surfactant and/or solvent may be used to disperse nanotubes in the polymer. Small amounts of the surfactant and/or solvent may be present in the final composition from which wall 33 is formed; however such amounts may be sufficiently small that the weight percent of the nanotubes relative to the polymer is effectively the same as the weight percent of the nanotubes relative to the entire composition. The various exemplary weight percentages listed above, unless otherwise noted, therefore apply to all different nanotube-polymer compositions provided herein, e.g., compositions consisting essentially of nanotubes and polymer, as well as compounds that contain low levels of other compounds, such as solvents and surfactants.

In some embodiments, the dilute nanotube-polymer composition also includes a lubricant. As is known in the art, lubricant may be added to nanotube-polymer compositions in order to improve dispersion of the nanotubes within the polymer. In other embodiments, the dilute nanotube-polymer composition excludes (i.e., contains no) lubricant. For example, depending on the particular application, the presence of lubricant may carry too high a risk of contamination (which can be detrimental in biological applications) and/or too high a risk of structurally weakening wall 33, and thus may be avoided. Other compounds optionally may be added to the composition, including pigments, plasticizers, dispersants, surfactants, contrast agents, and/or stabilizers. In some embodiments, each optional additive is present at a concentration of 1 wt. % or less in the composition.

In some embodiments, nanotubes 31 are randomly oriented throughout flexible wall 33 of balloon 30. That is, each individual nanotube may have an unconstrained orientation both relative to balloon 30 as a whole, through the thickness of wall 33, and relative to other nanotubes. Nanotubes 31 may form an at least partially interconnected web throughout flexible wall 33, so that at least some nanotubes 31 contact at least one other nanotube 31. Such contact may include a “crossing” contact in which the nanotubes cross at an angle of greater than zero degrees and less than or equal to 90 degrees. Alternatively, or additionally, such contact may include a “parallel” contact in which two or more nanotubes contact each other along at least a portion of their length (e.g., run in parallel contact for 5 or more nanometers). Those of skill in the art will recognize that such parallel contact between nanotubes 31 naturally arises due to attractive van der Waals forces, and may be referred to for the extent of that contact as “nano-ropes.” While in many embodiments a plurality of nanotubes 31, or even a majority of nanotubes 31 (e.g., more than half of nanotubes 31) are not aggregated in the form of nanoropes but are instead individually dispersed throughout wall 33, in other embodiments a majority of nanotubes 31 may be present predominantly in the form of nanoropes.

The interconnectedness of nanotubes within wall 33 affects the mechanical properties of balloon 30, so it may be useful both to control and characterize such interconnectedness. For example, in some embodiments, a majority of nanotubes 31 contact at least one other nanotube 31, while in other embodiments, a majority of nanotubes 31 do not contact any other nanotubes 31, and in still other embodiments, substantially no nanotubes 31 contact other nanotubes 31, that is, each nanotube 31 is substantially isolated from each other nanotube by the polymer in wall 33. Such interconnectedness of nanotubes 31 within wall 33 can be characterized using a variety of techniques, including microscopy (e.g., SEM or TEM), and electrical conductivity.

For example, the electrical conductivity of wall 33 depends, in part, on (a) the concentration of nanotubes within wall 33, (b) the electrical characteristics of other material components in wall 33 (e.g., the polymer electrical characteristics), and (c) the interconnectedness of the nanotubes within wall 33. As described in greater detail below, parameters (a) and (b) may be controlled during fabrication by selecting the amount of nanotubes to add to the composition from which wall 33 is formed, and by selecting the type of polymer and other components added to the composition, respectively. In contrast, parameter (c) may be controlled during fabrication by selecting the extent to which the nanotubes are dispersed within the polymer, which affects the extent to which they may contact one another. As is known in the art, during an electrical conductivity measurement, current is caused to flow across a material. Within flexible wall 33, the greater the extent to which the nanotubes are interconnected, the more current will flow, resulting in a larger electrical conductivity; conversely, the lesser the extent to which the nanotubes are interconnected, the less current will flow, resulting in a smaller electrical conductivity. By holding parameters (a) and (b) constant, but varying the extent to which the nanotubes are dispersed in the polymer (c) and observing both the electrical conductivity and the mechanical characteristics of balloon 30, a correlation between the electrical conductivity and the mechanical characteristics of balloon 30—both of which relate to the interconnectedness of nanotubes 31—can be derived and used to develop balloons having still further enhanced mechanical characteristics.

The electrical conductivity of the flexible wall may be at least 3×10⁻¹⁶ S/cm, or at least 6×10⁻⁹ S/cm, or at least 1×10⁻⁴ S/cm. For example, some embodiments of compositions having between 0.01 wt. % and 0.1 wt. % of nanotubes may have an electrical conductivity of at least 3×10⁻¹⁶ S/cm. Or, for example, some embodiments of compositions having between 0.1 wt. % and 0.4 wt % of nanotubes may have an electrical conductivity of at least 6×10⁻⁹ S/cm. Or, for example, some embodiments of compositions having greater than 0.4 wt. % may have an electrical conductivity of at least 1×10⁻⁴ S/cm. Such compositions may also include a polar polymer. For further details, see U.S. Pat. No. 6,936,653, the entire contents of which are incorporated herein by reference.

In an alternative embodiment of balloon 30, designated 30″ in FIG. 2C, inflation lumen is disposed against wall 21″ of shaft 21. Port 22″ exists in flexible wall 33″, and inflation lumen 28″ may pass through port 22″ and terminate within balloon 30″. Alternatively, the distal end of inflation lumen 28″ may be affixed to flexible wall 33″ such that it provides communication through port 22″.

While FIGS. 2A-2C illustrate nanotubes 31 as having an unconstrained alignment relative to balloon 30, nanotubes 31 may alternatively be aligned relative to balloon 30, and help to inhibit one or more potential modes of tearing of balloon 30. For example, FIG. 2D illustrates an embodiment in which nanotubes 31′ are aligned longitudinally relative to balloon 30′. The longitudinal orientation of nanotubes 31′ helps to inhibit potential radial modes of tearing of balloon 30′. In another embodiment (not illustrated), nanotubes 31′ are aligned radially relative to balloon 30′, which helps to inhibit potential longitudinal modes of tearing of balloon 30′.

In the embodiment illustrated in FIG. 2D, at least a subset of nanotubes 31′ generally extend between a first end 35′ of balloon 30′ and a second end 36′ of balloon 30′. However, not all of nanotubes 31′ need extend the entire distance between first and second ends 35′, 36′; for example, some or all of nanotubes 31′ may be longitudinally oriented relative to balloon 30′ but may only extend a portion of the distance between first and second ends 35′, 36′. Even if no nanotubes 3 extend the entire distance between ends 35′ and 36′, wall 33′ is still reinforced. Nanotubes 31′ also need not be precisely oriented relative to balloon 30′. For example, the orientation of each individual nanotube 31′ may deviate by more than 1%, more than 2%, more than 5%, or even more than 30% from imaginary line 37′, which represents an orientation that is exactly longitudinal relative to balloon 30′. Radially aligned nanotubes may have similar length and/or alignment as described for longitudinally aligned nanotubes.

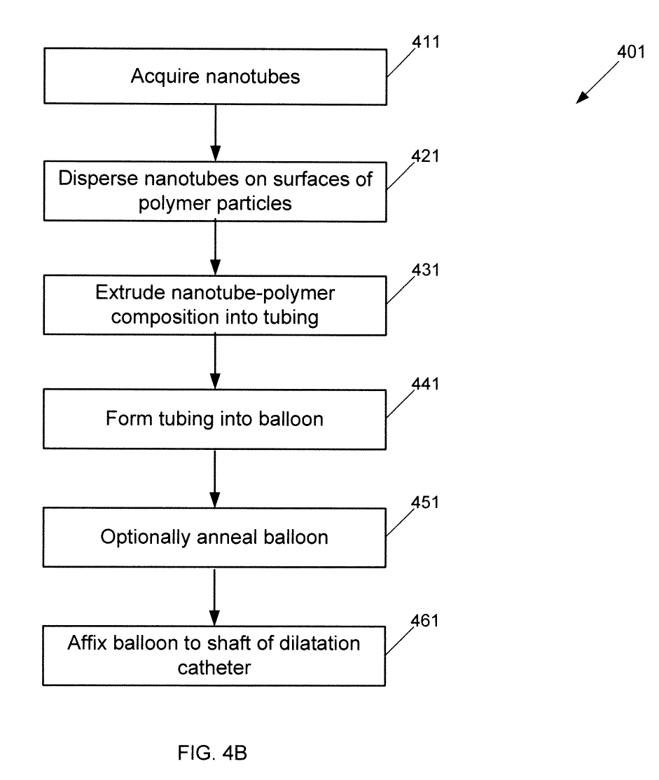

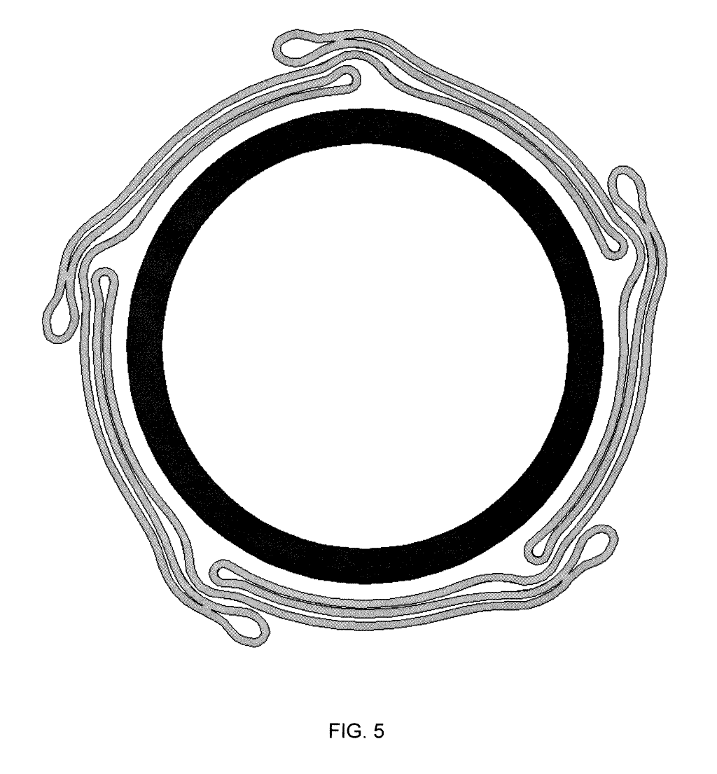

Nanotubes 31′ may be aligned longitudinally and/or radially relative to balloon 30′ using any suitable technique, e.g., gel spinning or electrospinning. In gel spinning, nanotubes 31′ are dispersed within a suitable melted polymer (e.g., as described below with respect to FIG. 4A). The melted nanotube/polymer mixture is extruded through a suitably shaped die and the extruded end drawn, which causes chains of the polymer and the nanotubes to align substantially parallel to the direction of extrusion. To form balloon 30′ having longitudinally oriented nanotubes 31′, the die is cylindrically shaped and includes a mandrel for forming a lumen within balloon 30′. To instead form balloon 30′ having radially oriented nanotubes 31′, the die is shaped to extrude a sheet or ribbon; after extrusion, the sheet or ribbon may be looped into a cylinder and the edges sealed (e.g., with heat and pressure) to form balloon 30′. Electrospinning includes applying an electrical field during extrusion that orients the nanotubes.

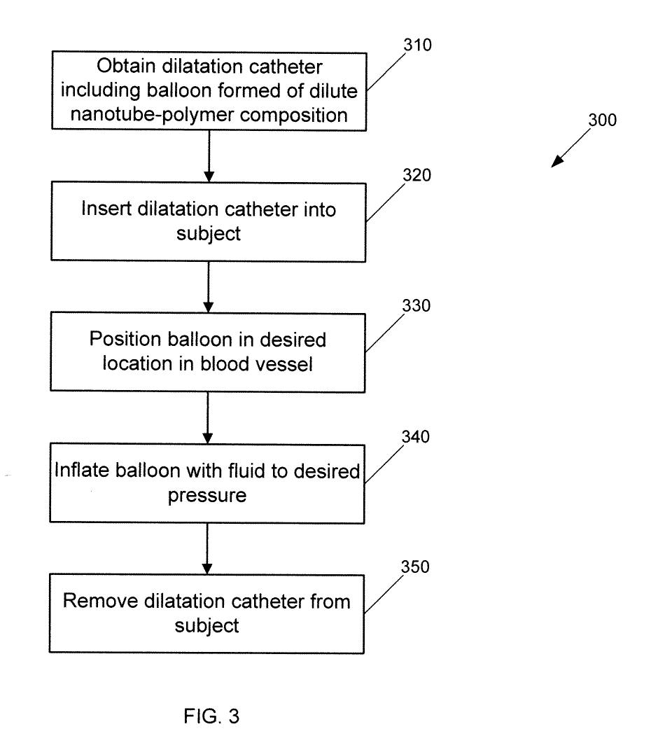

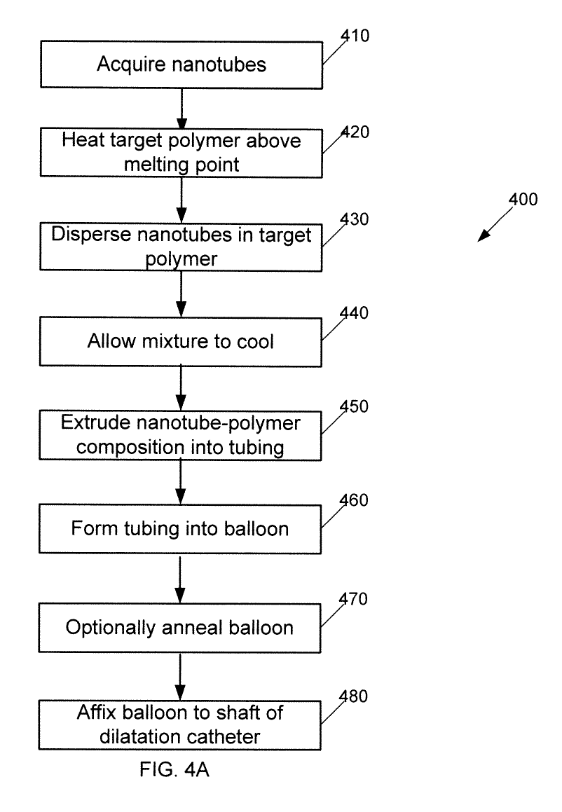

FIG. 3 illustrates steps in a method 300 of restoring patency to an occluded blood vessel using a dilatation catheter including a balloon formed using a dilute nanotube-polymer composition, according to some embodiments of the present invention. First, a dilatation catheter constructed in accordance with embodiment of the present invention which includes a balloon formed of a dilute nanotube-polymer composition is obtained (310).

The dilatation catheter is inserted into the subject (320). For example, the dilatation catheter may be inserted into a blood vessel (e.g., vein or artery) of the subject. The balloon is then positioned at a desired location in the blood vessel (e.g., adjacent a stenotic lesion) (330). For example, the balloon may be partially inflated with a fluid containing a contrast agent and imaged using conventional radiography in order to aid in positioning the balloon.

The balloon of the dilatation catheter then is inflated with the fluid to a desired pressure (340). The pressure may be sufficient to disrupt a stenotic lesion, while at the same time inflating the balloon to a diameter that is sufficient to expand, but not over-expand, the blood vessel. The dilatation catheter then is removed from the subject (350).

In some embodiments, the pressure to which the balloon is inflated is in the range of about 4 to 16 atm, or about 4 to 8 atm, or about 4 to 6 atm, or about 6 to 8 atm, or about 8 to 16 atm, or about 8 to 12 atm, or about 10 to 12 atm, or about 12 to 16 atm, or about 16 to 18 atm, or about 10 to 35 atm, or 10 to 20 atm, or 15 to 25 atm, or 20 to 30 atm, or 30 to 35 atm.

FIGS. 4A-4B illustrate exemplary methods of forming the balloon 30 of FIGS. 2A-2C, according to some embodiments.

Referring first to method 400 of FIG. 4A, nanotubes are acquired (410), for example from commercially available sources, or are fabricated. The nanotubes may include single-walled carbon nanotubes (SWNT) and/or multi-walled carbon nanotubes (MWNT). The carbon nanotubes may include conducting and/or semiconducting nanotubes. The carbon nanotubes may be “pristine,” that is, not functionalized, derivitized, or otherwise modified (e.g., including substantially no atoms other than carbon). Alternatively, one or more of the carbon nanotubes may be functionalized or derivitized to have desired therapeutic properties, or to enhance their dispersion in the polymer. In one example, the nanotubes are XD nanotubes (Unidym Corp., Menlo Park, Calif.), such as XD34 nanotubes, which are characterized as being “conductivity grade,” having a high percentage of SWNTs, and having an impurity level of 5% or less. In another example, the nanotubes are XO nanotubes (also from Unidym Corp.) which are characterized as being “fiber grade,” having a mix of MWNTs and SWNTs, and having an impurity level of 3% or less. In another example, “C-grade” MWNTs (NanoTech Labs, Yadkinville, N.C.) are characterized as being produced by Chemical Vapor Deposition (CVD), having diameters between 20-30 nm, and having a purity of greater than 95%. In the examples provided below, XD34 nanotubes were used as obtained from Unidym Corp. without further treatment. Other types of nanotubes are also suitable. Typically the length, diameter, type, level of impurity, and type of impurity (e.g., presence of amorphous carbon and/or residual catalyst) of the nanotubes are selected to enhance the mechanical strength of the flexible wall to be formed using those nanotubes. Without wishing to be bound by a theory, it is believed that nanotubes having a selected impurity type and impurity concentration may reduce the agglomeration of nanotubes into nanoropes, thus enhancing the dispersion of nanotubes in the polymer and by extension enhancing the strength of the flexible wall subsequently formed using those nanotubes.

In one embodiment, XD34 nanotubes from Unidym Corp. were characterized using techniques known in the art. It was found that there was substantial variation in the measured lengths with lengths ranging from 200 to over 7000 nm with the mean length being 1448 nm; the standard deviation of 1391 nm indicates that there is a wide variety of measured tube lengths. Diameters of the measured nanotubes were found to range in value from 1.5 nm to over 8 nm with a mean diameter of 3.77 nm and a standard deviation of 1.45 nm. The bulk of the nanotubes were double- or triple-walled (42% each), with the next most populous group being 4-WNT's (6%); single-, 5- and 6-WNT's were each found to be about 3% of the total population of tubes. The XD34 nanotubes were also found to contain a significant amount of non-tubular carbon species, specifically, disordered graphite and graphitic shells; the exact ratio of tubular to non-tubular species could not be measured. However it is believed that the impurities are less than 5%.

Other types of materials may be used in the balloon, either in addition to the nanotubes, or instead of the nanotubes. For example, inorganic nanotubes (e.g., tungsten disulfide, boron nitride, silicon, titanium dioxide, molybdenum disulfide, copper, or bismuth nanotubes) also may be used to reinforce the flexible wall of the balloon. Or, for example, graphene fibers may be used to reinforce the flexible wall of the balloon. Or, for example, fibers of KEVLAR® (poly paraphenylene terephthalamide), TEFLON® (polytetrafluoroethylene), TERLON® (poly(p-phenylenebenzobisthiazole)), ZYLON® (poly(p-phenylene-2,6-benzobisozazoles), polyether block amides, and VECTRAN® (liquid crystal polymer) may be used to reinforce the flexible wall of the balloon. The selected material(s) strengthen the balloon analogously to the nanotubes, e.g., as described above.

A suitable polymer is then heated above its melting point (420). Suitable polymers include elastomers such as EPDM, epichlorohydrin, nitrile butadiene elastomers, and silicones, epoxies, fluoropolymers such as polytetrafluoroethylene (PTFE, trade name TEFLON®), isocyanates, nylon, poly(acrylic acid), polyamides such as PEBAX® (tradename for polyether block amide, available from Arkema, Colombes, France), polybutene, polybutylene naphthalate, polycaprolactone, polycarbonate, poly(dimethylsiloxane), polyester, polyether, polyethylene, polyethylene naphthalate, polyethylene terephthalate (PET, trade name DACRON®, DuPont, Wilmingon Del.), polyimides, polyisobutene, polyisoprene, poly(methacrylic acid), polyolefin, polyoxide, polypropylene, polysiloxane, polystyrene, polysulfide, polyurea, polyurethane, poly(vinyl acetate), poly(vinyl alcohol), poly(vinyl chloride) (PVC), poly(vinyl pyridine), poly(vinyl pyrrolidone), urethanes, and copolymers thereof and combinations thereof, or other polymer that is biocompatible, in which nanotubes disperse, and that when mixed with nanotubes is capable of being formed into a balloon having desired mechanical properties. Examples of some suitable polymers may be found in U.S. Pat. No. 5,871,468, entitled “Medical Catheter With a High Pressure/Low Compliant Balloon,” the entire contents of which are incorporated herein by reference. Copolymers of tetrafluoroethylene with ethylene, cholorotrifluoroethylene, perfluoroalkoxytetrafluoroethylene, or fluorinated propylenes such as hexafluoropropylene also may be used. In some embodiments, the polymer is polar, or is at least partially polar, which in some embodiments may aid in dispersing the nanotubes throughout the polymer. For further details, see U.S. Pat. No. 6,936,653, the entire contents of which are incorporated herein by reference. In one example, the polymer includes, or consists essentially of, nylon, e.g., PA-12 (such as GRILAMID-L25®, available from EMS-Chemie AG, Reichenauerstrasse Switzerland, or L1800®, available from Evonik Industries, Essen Germany). In another example, the polymer includes, or consists essentially of, PEBAX®. In another example, the polymer includes, or consists essentially of, polyurethane.

The nanotubes then are mixed into the melted polymer while the polymer is maintained at a temperature above its melting point, forming a nanotube-polymer solution (430). Specifically, the nanotubes are added to the polymer in an amount sufficient for the balloon eventually formed using the polymer-nanotube mixture to have sufficient mechanical properties to withstand pressurization. For example, the nanotubes may be added to the polymer in an concentration of less than 0.5 wt. %, or less than 0.2 wt. %, in the composition. In one example, the nanotubes are added to the polymer in a concentration of between 0.2 wt % and 0.01 wt. % in the composition. In another example, the nanotubes are added to the polymer in a concentration of between 0.2 wt. % and 0.05 wt. % in the composition. In still another example, the nanotubes are added to the polymer in a concentration of between 0.15 wt. % and 0.05 wt. % in the composition. In some embodiments, no other materials are added to the composition other than the nanotubes and the polymer. In other embodiments, at least one other material (such as a lubricant, solvent, or surfactant) that aids dispersion of the nanotubes in the polymer is added. Typically, it is useful to reduce or avoid the presence of particulates in the composition because such particulates can reduce the mechanical strength and reliability of the balloon fabricated from the composition. In some embodiments, the nanotubes and/or polymer are selected to be of sufficient purity that substantially no particulates are present that would otherwise reduce the mechanical strength and reliability of a balloon formed from the composition.

The nanotubes may be thoroughly and substantially evenly distributed (dispersed) throughout the polymer using mechanical agitation, for example, using a material compounder, moving a container holding the nanotube-polymer solution, stirring the nanotube-polymer solution, or by maintaining the nanotube-polymer solution above the polymer melting temperature for an extended period of time, for example. The mechanical agitation can alternately be performed using sonic energy, e.g., using an ultrasonic homogenizer (sonicator), or using low-frequency high-energy sonic energy, such as a ResonantAcoustic® mixer available from Resodyn Inc., Butte Mont.

Once the nanotubes are distributed in the polymer to a satisfactory degree, the nanotube-polymer solution is allowed to cool (440), forming a dilute nanotube-polymer composition. The polymer may be cross-linked at this point, or at another suitable point, using electromagnetic (e.g., e-beam) radiation.

The composition is then extruded into tubing (450), which is then formed into a balloon (460), for example, as described below with respect to FIG. 4B. It should be understood that prior to forming the balloon, the nanotube-polymer composition is optionally pelletized and re-extruded or otherwise formed using known techniques.

The balloon is then optionally annealed (470), which may increase the burst pressure of the balloon. In one example, the balloon is annealed by raising the temperature of the balloon to a controlled level below the melt temperature of the composition, but high enough to give the polymer molecules the ability to move slightly. For a composition containing PA-12, the balloon may be held at a temperature of 120-180° F. for 30-120 minutes. Without wishing to be bound by a theory, it is believed that the annealing process relieves some of the residual stresses from balloon fabrication (e.g., from blow-molding) and allows some residual polymeric crystallization to take place, which may be a strengthening mechanism. Annealing may provide the balloon with an additional 5-15% increase in burst pressure and a reduction in the variation of the mechanical properties of the balloon (e.g., a reduced standard deviation in the burst pressure and the compliance of the balloon).

The nanotube-reinforced balloon then is affixed to the shaft of an interventional device such as a dilatation catheter (480), for example dilatation catheter 20 illustrated in FIG. 2A, using conventional affixation methods.

Optionally, other parts of the dilatation catheter may be formed of the dilute nanotube-polymer composition. For example, the composition may be used to form shaft 21 of dilatation catheter 20 illustrated in FIG. 2A. Nanotubes, if used in shaft 21, may provide increased axial strength and pushability to dilatation catheter 20, thereby allowing shaft 21 to be produced with reduced dimensions, while at the same time reducing the likelihood of kinking, binding, or similar problems that typically accompany the reduction in dimensions of dilatation catheter parts. Alternately, shaft 21 can be made with the same dimensions and having enhanced axial strength and pushability as compared to a shaft of the same dimensions but lacking nanotubes. Or, for example, the nanotubes may provide “steerability” to shaft 21 and/or guide wire 26. For further details, see U.S. patent Ser. No. 11/267,226, filed Nov. 3, 2005 and entitled “Radiopaque-Balloon Dilatation catheter and Methods of Manufacture,” the entire contents of which are incorporated by reference herein. The type of nanotube and the type of polymer used in balloon 30, and in other parts of dilatation catheter 20, may be selected independently of one another.

In embodiments in which both nanotube-reinforced balloon 30 and shaft 21 are made using a dilute nanotube-polymer mixture, the nanotubes may be used to enhance bonding between balloon 30 and shaft 21. Specifically, nanotubes have a propensity to lock together when brought near each other. Nanotube-reinforced balloon 20 and shaft 21 may be bonded together using a technique that allows the nanotubes in the two components to lock together, for example, by bringing the two components adjacent each other and then heating the polymer/nanotube composition just higher than the melting point of the polymer. While the polymer is heated, nanotubes in one component may move through the composition and lock together with nanotubes in the other component. Electromagnetic fields optionally may be used to selectively orient the nanotubes and/or enhance the transport of nanotubes from one component to the other.

FIG. 4B illustrates an alternative method 401 of forming a balloon using a dilute nanotube-polymer composition, such as balloon 30 illustrated in FIGS. 2A-2C.

Nanotubes are acquired (411), e.g., as described above with reference to FIG. 4A.

The nanotubes are then dispersed on the surfaces of solid polymer particles (421). For example, the polymer may be in the form of pellets, granules, grains, beads, microcapsules, microspheres, nanospheres, microparticles, micropellets, nanoparticles, or a powder (collectively referred to herein as “particles”). The particles may have sizes ranging between, for example, 1 nm and 250 μm, e.g., 10 nm to 100 μm, or 100 nm to 10 μm, or 10 μm to 250 μm. Sizes larger than 250 μm may also be used, for example, between 250 μm and 5 mm. e.g., 1 mm to 5 mm. In one example, a polymeric powder having particle sizes between 1 μm and 100 μm is used, e.g., having particle sizes between 5 and 60 μm. Without wishing to be bound by a theory, it is believed that small polymeric particle sizes can enhance the dispersion of nanotubes in the composition by increasing the available surface area to which the nanotubes may adhere, and thus reducing the likelihood that the nanotubes may agglomerate together into nanoropes.

The polymer may be one of the polymers listed above with reference to FIG. 4A. In one example, the polymer includes, or consists essentially of, nylon, e.g., PA-12. In another example, the polymer includes, or consists essentially of, PEBAX®. In another example, the polymer includes, or consists essentially of, polyurethane. The polymer may be cross-linked using electromagnetic (e.g., e-beam) irradiation to modify its properties.

The nanotubes may be dispersed on the surfaces of the polymer particles using several suitable techniques, some examples of which are provided below.

In some embodiments, the nanotubes are added in a solid state (e.g., as a powder) directly to the particulate polymer, and the mixture then mechanically agitated to coat the outer surfaces of the polymer particles with nanotubes. Such mechanical agitation can include, for example, manually moving a container holding the nanotube-particulate polymer mixture, using a material compounder, or stirring the nanotube-particulate polymer mixture. The mechanical agitation can alternately be performed using sonic energy, e.g., using an ultrasonic homogenizer (sonicator), or using low-frequency high-energy sonic energy, such as a ResonantAcoustic® mixer available from Resodyn Inc., Butte Mont.

In other embodiments, the nanotubes are added in a solubilized state (e.g., dispersed in a suitable solvent and/or surfactant) to the particulate polymer. For example, the liquid state nanotubes may be sprayed onto the surface of the polymer particles, and the liquid subsequently removed to leave the nanotubes on the surface of the polymer particles. The polymer particles may be stationary while the nanotubes are applied to them, or they may be agitated (e.g., tumbled) while the nanotubes are applied to them. Or, for example, the polymer particles are added to a liquid containing the nanotubes, the mixture agitated (e.g., as described above), and the liquid then removed to leave the nanotubes on the surface of the polymer particles. In such embodiments, the liquid can be removed in any suitable manner, for example, by heating the mixture to drive off the liquid, or allowing the liquid to evaporate at ambient temperature. The mixture subsequently may be “powderized” to separate the nanotube-coated particles from one another, thus inhibiting agglomeration of nanotubes. Powderizing may be performed by mechanical agitation, e.g., by sonication or other agitation mechanism described above. Alternatively, the solution of nanotube-coated particles may be spread (e.g., by spraying) onto a surface to enhance evaporation of the liquid while simultaneously inhibiting agglomeration of nanotubes.

In still other embodiments, the nanotubes are added in a gaseous state to the particulate polymer. For example, the nanotubes can be aerosolized, and the polymer particles exposed to the aerosol, e.g., by spraying the polymer particles into a container holding the aerosolized nanotubes. The nanotubes are attracted to and adhere to the surfaces of the polymer particles, which subsequently may be collected, e.g., by allowing them to fall to the bottom of the container.

The polymer optionally may be softened (e.g., using heat or a suitable solvent) to enhance adhesion of the nanotubes to the surface of the polymer particles. The composition of the polymer and/or the surface characteristics of the polymer particles may also be selected to enhance adhesion of the nanotubes to the surface of the polymer particles. The size of the particles may also be selected to (a) enhance the attraction of the nanotubes to the particle surfaces, and (b) enhance the interconnectedness of the nanotubes within the composition. The parameters (a) and (b) may not necessarily depend on the particle size in the same way, so the particle size may be selected based on a balance between (a) and (b) for the desired purpose of the composition.

In some embodiments, a multi-step process may optionally be used to first encapsulate nanotubes with a thin layer of a first polymer, and the coated nanotubes then mixed with a second polymer that may be the same or different from the first polymer. For example, a polymer can be aerosolized, and the nanotubes exposed to the aerosol, e.g., by spraying the aerosolized polymer particles into a container holding the nanotubes. The polymer-coated nanotubes may then be mixed with the same or a different polymer. For example, nylon-coated nanotubes may be dispersed in PEBAX®. For further details on certain methods of coating nanotubes with polymers, see the following patent references, the entire contents of each of which are incorporated herein by reference: U.S. Pat. No. 7,264,876 and U.S. Pat. No. 7,008,563.

After the nanotubes are dispersed on the surfaces of the polymer particles, the particles are extruded into tubing formed from the dilute nanotube-polymer composition (431). The particles may be directly extruded into the tubing, or may be first pelletized and the pellets extruded into the tubing.

In an exemplary extrusion process, the mixture of particulate polymer and nanotubes is loaded into a heated, barrel-shaped container to dry them. The mixture falls into an extruder and as the polymer particles are heated above their melting point they melt and liquefy, and a rotating screw in the extruder mixes the polymer and dispersed nanotubes into a substantially homogeneous blend. The liquefied mixture is then pumped through the extruder, which has a nozzle at one end, with an airtube centered in the nozzle. The airtube assists in controlling the finished dimensions of the tubing. The liquefied mixture exits the extruder as a long tube, the outside diameter of which is defined by the die diameter, and the inside diameter of which is defined by the airtube. The tube is pulled from the nozzle and through a cooling bath by a mechanical puller, thus solidifying the tubing. The tubing may be cut to individual lengths. In one example, the particles are extruded using a conventional extruder, such as a Microtruder (Randcastle Extrusion Systems, Inc., Cedar Grove, N.J.).

The tubing is then formed into a balloon (441). In one embodiment, each balloon is formed from an individual length of extruded tubing using a two-step process. The first step is necking the tube into a balloon parison. During this parison process, the tubing is heated and stretched. The heating is controlled below the melting point, such that it lowers the yield strength for the localized material that is being stretched.

The second step of balloon formation is blow-molding. The balloon parison is inserted into a heated mold that is mounted in a balloon blow machine. There are various molds that correspond to different finished diameters and lengths of balloons; the particular mold is selected based on the desired characteristics of the finished balloon. Next, one end of the balloon parison is clamped shut, and the open end is connected to a supply of compressed nitrogen or other non-reactive gas. The compressed nitrogen is then actuated, which pressurizes the balloon parison to a constant internal pressure while the heated mold warms the parison. The balloon blow machine includes one or more sensors that determine when the parison has reached a suitable temperature for the next step of blow-molding. After the warm-up, the parison may be stretched slightly by the pressure of the compressed nitrogen. The nitrogen pressure is then increased to a higher pressure for a specified amount of time. During this time, the heated parison stretches to conform to the shape of the mold, thus forming the balloon. The balloon is then cooled and removed from the mold.

The balloon thus formed has flexible walls that are formed of the dilute nanotube-polymer composition and have a selected thickness. The wall thickness is selected based, in part, on the desired rated burst pressure and the inflated balloon diameter. For example, “workhorse” balloon catheters intended for coronary use may have a rated burst pressure between 12-18 ATM and an inflated diameter between 2.5 mm and 4.0 mm.

Conventionally, such coronary balloons may have a wall thickness of 0.0004″ to 0.0010,″ while conventional balloons intended for peripheral use may be fabricated having a wall thickness of 0.0008″ to 0.0015″. Because the compositions provided herein provide balloons of enhanced mechanical strength, thinner walls than conventionally possible can now be fabricated, thus providing balloons with improved burst strength but having a significantly smaller cross-sectional profile in the collapsed state.

For example, the hoop stress (HS) for a balloon may be expressed by the equation:

HS=PD/2T

where P is the balloon burst pressure, D is the balloon diameter, and T is the balloon wall thickness. Thus, for a given balloon material, the wall thickness is directly proportional to the burst pressure. As illustrated by the examples provided further below, the use of a dilute nanotube-polymer composition may increase the strength of a balloon by 15% relative to an otherwise similar balloon lacking nanotubes. This increased strength allows the thickness of the balloon to be reduced proportionally while maintaining the same burst pressure.

In one example, assuming that the composition improves the burst pressure of a balloon having a 0.0005″ wall thickness by 15%, then the appropriate redesigned wall thickness to maintain the same burst pressure may be calculated as follows:

P₁/T₁=P₂/T₂

P₁/T₁=(1.15P₁)/T₂

T₁=0.0005″/1.15

T₁=0.00043″

where P₁ is the burst pressure of the redesigned balloon, T₁ is the wall thickness of the redesigned balloon, P₂ is the burst pressure of the original balloon (formed using the dilute nanotube-polymer composition), and T₂ is the wall thickness of the original balloon. Thus, a balloon having a single 0.0005″ wall may be redesigned to instead have a 0.00043″ wall using such a composition. When such a redesigned balloon is mounted on a catheter and folded, the cross-sectional profile may be reduced by about 0.0005″ to 0.0015″. FIG. 5 is a cross-sectional illustration of a folded balloon mounted on a catheter shaft. Note that there are multiple alternative ways of folding balloons. Typically, because such folding involves overlapping layers of the flexible balloon walls, reducing the dimensions of such walls can lead to a reduced cross-sectional diameter of the folded balloon.

As discussed above with reference to FIG. 4A, the balloon optionally may be annealed (451) to further enhance its mechanical characteristics. The balloon is then affixed to the shaft of an interventional device such as a dilatation catheter (461).

Other methods can also be used to form the dilute nanotube-polymer composition and/or the balloon. For example, nanotubes may be dispersed in a monomer (and optionally also a solvent), and the monomer polymerized to form the dilute nanotube-polymer composition. The composition may then be formed into a balloon, e.g., as described above.

Examples of suitable blood vessels for treatment using the balloons provided herein include renal, iliac, femoral, distal leg, coronary and carotid arteries as well as saphenous vein grafts, synthetic grafts and arterioveinous shunts used for hemodialysis. It is contemplated that the balloons described herein have applicability for use with any other type of body passageway, including, but not limited to, urethra, prostate, prostatic urethra, esophagus, fallopian tubes, rectum, intestines, bronchi, kidney ducts, wind pipe, pancreatic ducts, gall bladder ducts, biliary ducts, brain parenchyma, and the like.

The term “contrast agent” refers to a biocompatible radiopaque material capable of being monitored during injection into a subject by, for example radiography. The contrast agent may be either water soluble or water insoluble and in some embodiments does not contain radioactivity above the native or endogenous amounts naturally occurring in the elements employed. Examples of water soluble contrast agents include metrizamide, iopamidol, iothalamate sodium, iodomide sodium, and megalumine. Examples of water insoluble contrast agents include tantalum, tantalum oxide, and barium sulfate, each of which is commercially available in the proper form for in vivo use including a particle size of about 10 μm or less. Other water insoluble contrast agents include gold, tungsten, and platinum powders.

### EXAMPLES

Some non-limiting examples of the improved characteristics of exemplary balloons formed using dilute nanotube-polymer compositions will now be provided.

Three example compositions were prepared using the procedure set forth in FIG. 4B. A first composition “A” was prepared by manually agitating 0.10 wt. % of single-walled nanotubes (SWNT) (XD34 from Unidym Corp., Menlo Park Calif.) with 99.9% nylon (PA-12, L1800 from Evonik Industries, Essen Germany) for 1-2 minutes; and subsequently extruding and pelletizing the mixture by starve-feeding the compounding screw of a Randcastle Microtruder (vertical compounding screw set-up). A second composition “B” was identically prepared but included 5.0 wt % of the same SWNT and 95.0% of the same PA-12. A third composition “C” was identically prepared using 100% PA-12, and did not include any nanotubes (control).

The extruded and pelletized compositions A, B, and C were then extruded into tubing having an inner diameter of 0.21″ and an outer diameter of 0.33″. The tensile strength of the tubing was tested using an in-house procedure using ASTM D538-08 or ISO 527-1 and ISO 527-2 as a guideline. The tubing formed from compositions A and B were found to have a higher yield strength than the tubing formed from composition C. It was observed that the tubing formed from composition A was relatively smooth.

It was attempted to blow mold the tubing formed from compositions A, B, and C into balloons of 3 mm diameter and 19 mm length. The tubing formed from compositions A and C were readily blow molded into a balloon having the desired dimension. It was noted that a slightly higher pressure was required to blow mold the tubing formed from composition A than for the tubing formed from composition C. It was not possible to blow mold the tubing formed from composition B into a balloon. Specifically, it appeared that the nanotubes in the tubing formed from composition B were unevenly distributed, causing sufficient structural variations in the film that it could not be blown into a balloon. Without wishing to be bound by a theory, it is believed that too high a concentration of nanotubes relative to the balloon wall thickness may create defects or regions of increased stress that may lower the rated burst pressure.

The balloons formed from compositions A and C were pressure tested to rupture without annealing. As the data in Table 1 illustrates, the balloons formed from composition A demonstrated a significantly higher average burst pressure than those formed from composition C, and the standard deviation of the burst pressures for the balloons formed from composition A was notably lower than that of the balloons formed from composition C. The burst pressures and standard deviation values obtained for the balloons formed from composition C were well within the expected range for a conventional balloon of that composition. Thus, the balloons formed from composition A demonstrate significantly enhanced mechanical properties.

TABLE 1
Balloon burst pressures

| Composition from which | Average Burst Pressure | |
|---|---|---|
| balloon is formed | (ATM) | St. Dev. |
| Composition A (n = 3) | 16.28 | 0.07 |
| Composition C (n = 3) | 12.95 | 1.46 |

Balloons formed from compositions A and C were also annealed as described above with reference to FIG. 4A, and then pressure tested to rupture. As the data in Table 2 illustrates, the annealed balloons fanned from composition A demonstrated a significantly higher average burst pressure than those formed from composition C, and the standard deviation of the burst pressures for the balloons formed from composition A was notably lower than that of the balloons formed from composition C. The burst pressures and standard deviation values obtained for the annealed balloons formed from composition C were well within the expected range for a conventional balloon of that composition. Thus, the annealed balloons formed from composition A demonstrate significantly enhanced mechanical properties.

TABLE 2
Annealed balloon burst pressures

| | Composition A Annealed | Composition C Annealed |
|---|---|---|
| | Balloon (n = 3) | Balloon (n = 3) |
| | Burst Pressure (ATM) | Burst Pressure (ATM) |
| | 17.39 | 13.26 |
| | 17.53 | 15.55 |
| | 17.43 | 15.19 |
| Average | 17.45 | 14.8 |
| St. Dev. | 0.07 | 1.33 |

In another example, a composition “D” was prepared by repeating the procedure described above for the fabrication of a balloon using composition A, but using a different extruder to form the tubing from composition D. Table 3 summarizes the results of testing on the balloon formed using composition D, as compared to those formed using compositions A and C.

TABLE 3
Annealed balloon burst pressures for balloons
formed using different extruders

| Composition from which | Average Burst Pressure | |
|---|---|---|
| annealed balloon is formed | (ATM) | St. Dev. |
| Composition A (n = 3) | 17.45 | 0.07 |
| Composition D (n = 10) | 18.60 | 0.84 |
| Composition C (n = 3) | 14.8 | 1.33 |

It was observed that the mechanism by which the dilute nanotube-polymer composition is extruded may affect the burst pressure (and standard deviation) of the balloon formed using that tubing. This indicates that extrusion settings may be selected to enhance or optimize the mechanical characteristics and reliability of interventional devices formed using dilute nanotube-polymer compositions. Without wishing to be bound by a theory, it is believed that agglomerations of nanotubes in the composition may be further dispersed using certain extrusion settings, thus improving the distribution of nanotubes throughout the polymer.

Although various embodiments of the present invention are described above, it will be evident to one skilled in the art that various changes and modifications may be made without departing from the invention. It is intended in the appended claims to cover all such changes and modifications that fall within the true spirit and scope of the invention.

What is claimed:

1. An interventional device comprising a balloon having a flexible wall, the flexible wall comprising a composition comprising between 0.005 wt. % and 0.20 wt. % of carbon nanotubes dispersed in a polymer.

2. The device of claim 1, wherein the interventional device comprises an elongated shaft having proximal and distal ends and a lumen therebetween, wherein the balloon is affixed to the elongated shaft near the distal end.

3. The device of claim 1, wherein at least a subset of the nanotubes contact other nanotubes to form a reinforcing web through the flexible wall of the balloon.

4. The device of claim 1, wherein the nanotubes are randomly oriented relative to an orientation of the balloon.

5. The device of claim 1, wherein the nanotubes are substantially evenly dispersed throughout the composition.

6. The device of claim 1, wherein the polymer comprises one of nylon, PEBAX®, polyurethane, silicone, PET, and polyethylene.

7. The device of claim 1, wherein the wall has a thickness of less than 0.0005″.

8. The device of claim 1, wherein the wall has a thickness of less than 1/250 of a nominal diameter of the balloon.

9. The device of claim 7, wherein the balloon has a rated burst pressure of greater than 16 atm.

10. The device of claim 1, wherein the composition comprises between 0.05 wt. % and 0.2 wt. % of the nanotubes.

11. The device of claim 1, wherein the composition comprises between 0.1 wt. % and 0.2 wt. % of the nanotubes.

12. The device of claim 1, wherein the composition consists essentially of the polymer and the nanotubes.

13. A method of making an interventional device, the method comprising:
contacting a plurality of polymer particles with a plurality of nanotubes;
extruding the polymer particles and the nanotubes to form a composition comprising the polymer and the nanotubes; and
forming the composition into a balloon.

14. The method of claim 13, wherein the nanotubes are in the form of a powder, and wherein said contacting comprises agitating the polymer particles and the nanotubes together to adhere the nanotubes to surfaces of the particles.

15. The method of claim 13, wherein the nanotubes are suspended in a liquid, and wherein said contacting comprises spraying the liquid onto the polymer particles.

16. The method of claim 13, wherein the nanotubes are aerosolized, and wherein said contacting comprises spraying the polymer particles into the aerosol.

17. The method of claim 13, wherein the composition comprises between 0.005 wt. % and 0.2 wt. % of nanotubes.

18. The method of claim 13, comprising selecting a size of the polymer particles to provide a desired degree of interconnectedness of the nanotubes in the balloon.

19. The method of claim 13, wherein the composition consists essentially of the polymer and the nanotubes.

20. The method of claim 13, wherein the polymer comprises one of nylon, PEBAX®, polyurethane, silicone, PET, and polyethylene.

21. The method of claim 13, further comprising affixing the balloon to a shaft.

22. The method of claim 13, further comprising annealing the balloon.
